(19) United States
(12) Patent Application Publication
(10) Pub. No.: US 2006/0068431 A1
(43) Pub. Date: Mar. 30, 2006

(54) Micro RT-PCR apparatus and method using the same

(76) Inventors: Gwo-Bin Lee, Tainan City (TW); Chia-Sheng Liao, Taipei City (TW)

Correspondence Address: CHRISTIE, PARKER & HALE, LLP, PO BOX 7068, PASADENA, CA, 91109-7068, US

(21) Appl. No.: 11/227,838

(22) Filed: Sep. 14, 2005

(30) Foreign Application Priority Data
Sep. 27, 2004 (TW) 093129227

(51) Int. Cl. C12Q 1/68; C12P 19/34; C12M 1/34

(52) U.S. Cl. 435/6; 435/91.2; 435/287.2

(57) ABSTRACT

A micro RT-PCR apparatus includes a chip module and control module. The chip module includes a reaction chamber unit, and a heating unit for heating the reaction chamber unit. The control module controls the heating unit to perform a first heating operation and a second heating operation. The first heating operation provides the reaction chamber unit with a temperature required to carry out a reverse transcription reaction. The second heating operation provides the reaction chamber unit with a temperature required to perform a polymerase chain reaction. An RT-PCR method using the micro RT-PCR apparatus is also disclosed.

### CROSS-REFERENCE TO RELATED APPLICATION

This application claims priority from Taiwanese Patent Application No. 93129227, filed on Sep. 27, 2004.

### BACKGROUND OF THE INVENTION

1. Field of the Invention

The invention relates to an apparatus and method for carrying out reverse transcription polymerase chain reaction (RT-PCR) assays, more particularly to a micro RT-PCR apparatus through which reverse transcription-polymerase chain reaction (RT-PCR) assays can be carried out automatically, and a method of using the micro RT-PCR apparatus.

2. Description of the Related Art

Micro electro mechanical system technology has been utilized in the manufacture of miniature biomedical detection apparatuses, which are in demand due to the advantages of high efficiency, disposability, portability, low consumption of assay sample, reduced energy consumption and low cost.

Polymerase chain reaction (PCR) is a method commonly employed to amplify a target sequence for the detection of the presence of a DNA template. PCR generally includes the steps of denaturation, annealing and extension of a specific DNA with use of a pair of primers. Although there are many reports relating to researches on PCR chips, only two types of PCR chips have been used commonly for polymerase chain reaction (PCR) assays since 1933; one being a sample-moving type in which a sample is moved between several regions which are heated to different controlled temperatures, the other being a temperature-cycling type in which a sample is placed within a fixed region whose temperature is changed within a cycle temperature. These types of PCR chips are limited to analysis for the identification of DNA viruses and have deficiency, such as high energy consumption and uneven temperature elevation.

Reverse transcription-polymerase chain reaction (RT-PCR) is a known method for the detection of the presence of a specific RNA virus and is carried out by producing a DNA fragment complementary to RNA through a reverse transcriptase reaction, followed by a performing polymerase chain reaction using the complementary DNA as a template. A target sequence may be amplified up to several million times through the RT-PCR method. With such an amplification, RNA viruses, such as Dengue virus, Enterovirus, Severe Acute Respiratory Syndrome (SARS), if present, can be easily detected. However, apparatuses for the RT-PCR method available in the prior art have not been miniaturized. Therefore, development of a micro RT-PCR apparatus is desirable in order to conserve as much space as possible and to reduce costs of the apparatuses.

### SUMMARY OF THE INVENTION

An object of the present invention is to provide an RT-PCR apparatus which is miniaturized and through which a two-step reaction process of RT-PCR can be carried out automatically.

Another object of the present invention is to provide a micro RT-PCR apparatus which is efficient and which can be mass-produced easily at low cost.

According to one aspect of the present invention, a micro (RT-PCR) apparatus comprises: a chip module which includes a reaction chamber unit, and a heating unit for heating the reaction chamber unit; and a control module for controlling the heating unit to perform a first heating operation and a second heating operation. The first heating operation provides the reaction chamber unit with a temperature required to carry out a reverse transcription reaction. The second heating operation provides the reaction chamber unit with a temperature required to perform a polymerase chain reaction.

According to another aspect of the present invention, a RT-PCR method comprises: using a chip module which includes a reaction chamber unit and a heating unit for heating said reaction chamber unit; controlling said heating unit to perform a first heating operation and a second heating operation, said first heating operation providing said reaction chamber unit with a temperature required to carry out a reverse transcription (RT) reaction, said second heating operation providing said reaction chamber unit with a temperature required to perform a polymerase chain reaction (PCR); and conducting an RT reaction and a PCR reaction in said reaction chamber unit.

### BRIEF DESCRIPTION OF THE DRAWINGS

Other features and advantages of the present invention will become apparent in the following detailed description of the preferred embodiments with reference to the accompanying drawings, of which:

FIG. 1 is a perspective view of a chip module used in the first preferred embodiment of a micro RT-PCR apparatus according to the present invention;

FIG. 2 is a plan view of the chip module of FIG. 1;

FIG. 3 is a block diagram showing the chip module of FIG. 1 connected to a control module according to the present invention;

FIG. 4 is a plan view of a chip module used in the second preferred embodiment of this invention;

FIG. 5 is a plan view of a chip module used in the third preferred embodiment of this invention;

FIG. 6 is a block diagram showing the chip module connected to a control module in the third preferred embodiment;

FIG. 7 is a schematic view showing a female mold for forming an upper substrate part of the chip module; and

FIG. 8 is a picture run on the agarose gel, which shows a DNA product obtained by RT-PCR from Dengue virus, Lane 1 representing DNA marker, Lane 2 representing the RT-PCR amplified DNA fragment having 419 bp, and Lane 3 and 4 representing the DNA fragments by digestion of the amplified DNA fragment with Hind III and Bst II, respectively.

### DETAILED DESCRIPTION OF THE PREFERRED EMBODIMENTS

Before the present invention is described in greater detail, it should be noted that like elements are denoted by the same reference numerals throughout the disclosure.

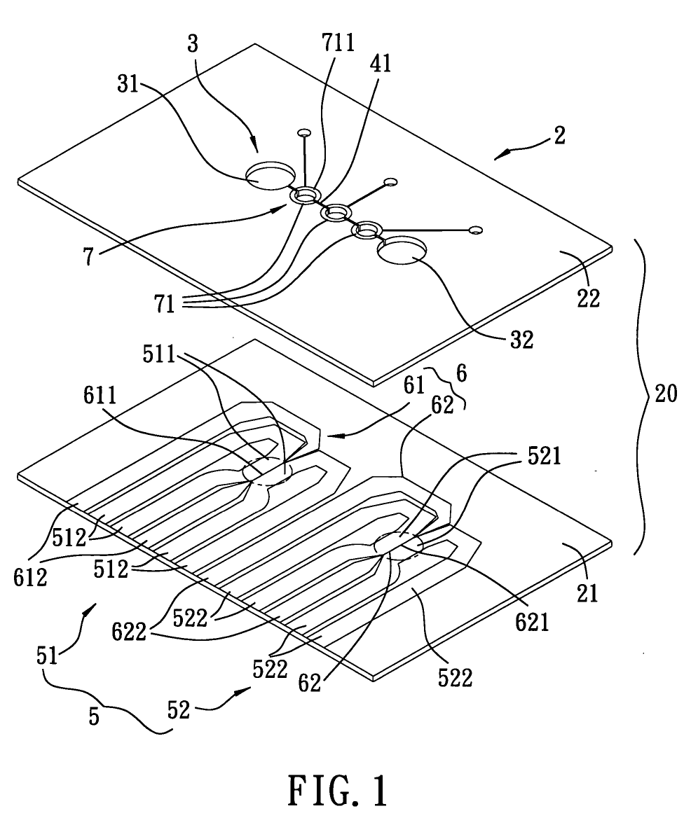

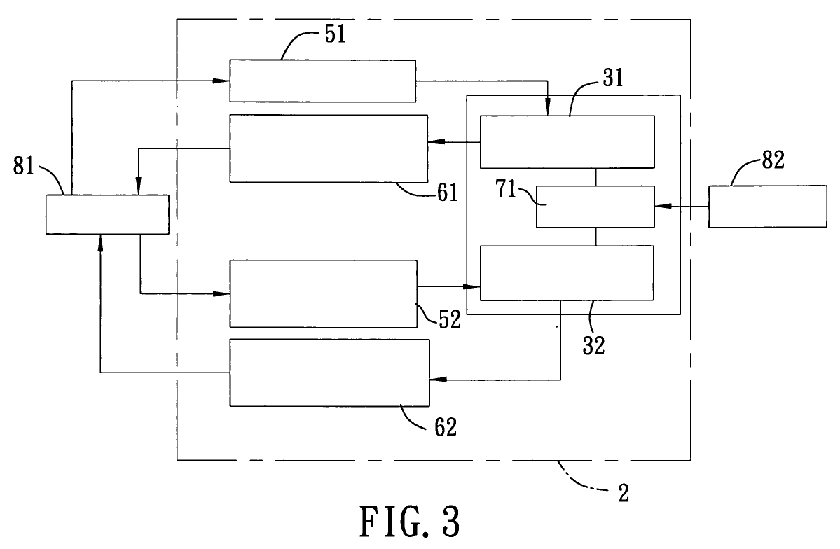

Referring to FIGS. 1-3, there is shown the first preferred embodiment of a micro RT-PCR apparatus according to the present invention which comprises a chip module 2 and a control module 8 (shown in FIG. 3). The chip module 2 comprises a substrate 20, and a reaction chamber unit 3 provided in the substrate 20. The reaction chamber unit 3 includes a RT chamber 31, a PCR chamber 32, and a chamber-to-chamber channel 41 communicated fluidly with the RT chamber 31 and the PCR chamber 32. A pump unit 7 is associated with the chamber-to-chamber 41 to control fluid transfer from the RT chamber 31 to the PCR chamber 32.

A heating unit 5 is provided in the substrate 20 and includes a first heating device 51 for heating the RT chamber 31 to a temperature needed to carry out a reverse transcription reaction, and a second heating device 52 for heating the PCR chamber 32 to a temperature needed to carry out the polymerase chain reaction. The first heating device 51 includes two heating elements 511, and two pairs of conductor traces 512 each pair of which is connected to one of the heating elements 511. Likewise, the second heating device 52 includes two heating elements 521, and two pairs of conductor traces 522 each pair of which is connected to one of the heating elements 521. These heating elements (511, 521) heat stably and evenly the respective RT chamber 31 and PCR chamber 32 to elevate the temperatures within the RT chamber 31 and the PCR chamber 32.

A temperature-sensing unit 6 is provided in the substrate 20 and includes a first temperature-sensing device 61 provided in the RT chamber 31 and a second temperature-sensing device 62 provided in the PCR chamber 32. The first temperature-sensing device 61 has a temperature-sensing element 611 for sensing the temperature of the RT chamber 31 and a pair of conductor traces 612 connected to the temperature-sensing element 611. Likewise, the second temperature-sensing device 62 has a temperature-sensing element 621 for sensing the temperature of the PCR chamber 32 and a pair of conductor traces 622 connected to the temperature-sensing element 621.

The pump unit 7 includes a chamber channel pump 71 which has three valves 711 that are provided in series in the chamber-to-chamber channel 41 and that are spaced apart from each other. Each valve 711 may include a membrane (not shown) operated by a pneumatic pressure to close or open the valve 711. The membranes of the valves 711 in the chamber-to-chamber channel 41 are controlled by the control module 8 to open and close sequentially with a time lag therebetween so that a predetermined amount of a sample may be transferred from the RT chamber 31 to the PCR chamber 32. Since the construction of the pump unit 7 including the valves 711 does not form any part of the present invention, it is not detailed hereinafter.

The control module 8 is a computerized control module and includes a temperature control unit 81 which controls the first and second heating devices 51 and 52 so that the first heating device 51 is activated to perform a first heating operation to raise the temperature of the RT chamber 31 to a level required for RT and so that the second heating device 52 is activated to perform a second heating operation to raise the temperature of the PCR chamber 32 to a level required for PCR. The temperature control unit 81 is connected to the first and second temperature-sensing devices 61 and 62 and the first and second heating devices 51 and 52 to control the temperatures of the RT and PCR chambers 31, 32 according to the signals received from the first and second temperature-sensing devices 61, 62.

The control module 8 further includes a pump control unit 82 to control the chamber channel pump 71 so that the valves 711 are actuated to open after the first heating operation of the first heating device 51. The pump control unit 82 may include a set of electromagnetically operated pneumatic valves which provide compressed air outputs to actuate the valves 711.

The control module may further includes a central processing unit connected to the temperature control unit 81 and the pump control unit 82 through an interface to monitor reaction process variables of the RT-PCR reaction. The reaction process variables would be the temperature of the RT and PCR chambers 31, 32, the heating time of the first and second heating devices 51, 52, the flow rate flowing through the chamber-to-chamber channel 41, etc.

The following is an example using the first embodiment of the present invention to conduct an analysis of a sample containing Dengue virus through the RT-PCR method. The RT-PCR conditions were set to be 42 degree C. (30 min) for RT and to be a temperature cycle for PCR, which passes through 95 degree C. (10 sec), 52 degree C. (15 sec) and 72 degree C. (30 sec). A pair of appropriate primers (primer seq.) was also selected for the analysis. A sample containing Dengue virus and an RT reagent were injected into the RT chamber 31. The control module 8 controlled the first heating device 51 to perform a first heating operation so that the RT chamber was heated by the first heating device 51 and kept at 42 deg. C. for 30 min and thereafter at 95 degree C. for 2 min. The first sensing device 61 sensed the temperature of the RT chamber 31 and transmitted signals to the temperature control unit 81 of the control module 8 to control the first heating device 51. A successful amplification of cDNA synthesized from the 10723-base Dengue-2 virus template was formed in the RT chamber 31. Afterward, the pump control unit 82 actuated the pump unit 7 so that the complementary DNA flowed into the PCR chamber 32 into which PCR reagents were injected for reaction with the complementary DNA. At the same time, the temperature control unit 81 controlled the second heating device 52 to perform a second heating operation during which the second heating device 52 heated the PCR chamber 32 so that the temperature cycle in the PCR chamber 32 passes through 95 deg. C. for 10 sec, 52 deg. C. for 15 sec, and 72 deg. C. for 20 sec in each cycle. The number of times that the temperature cycle occurs is thirty. A DNA fragment having 419 bp for encoding the non-structural protein (NS1) was therefore amplified.

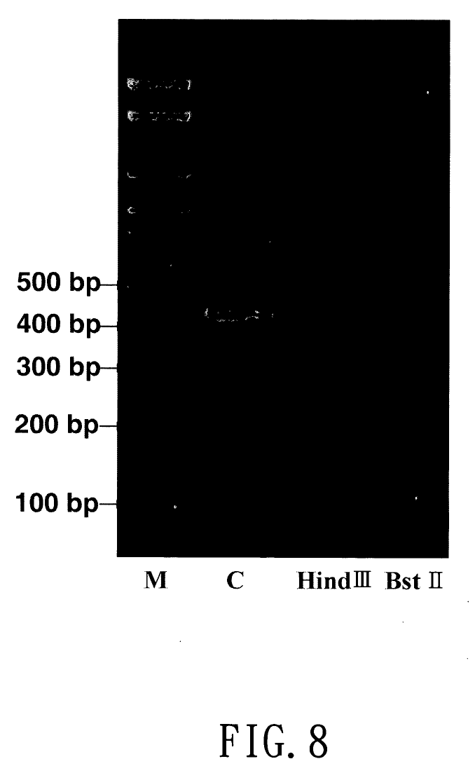

The NS-1 encoding DNA was further digested by restriction enzyme Hind III to form two fragments having 193 bp and 226 bp, respectively, and by restriction enzyme Bst II to form two fragments having 187 bp and 232 bp, respectively. The fluorescence signal of DNA products ran on agarose gel and was shown in FIG. 8, in which Lane 1 represents a DNA marker, Lane 2 represents the RT-PCR amplified DNA fragment having 419 bp for encoding the non-structural protein (NS1), Lane 3 and 4 represent the DNA fragments formed by digestion of the amplified DNA fragment with Hind III and Bst II, respectively.

Compared with the conventional RT-PCR apparatuses, in which the temperature is elevated at a rate of 2° C./sec and is decreased at a rate of 1° C., the temperature of the chip module 2 according to this invention can be increased at a rate of 20° C./sec and decreased at a rate of 10° C./sec. Thus, an RT-PCR analysis can be accomplished within a shorter period, e.g. 60 min. Furthermore, since the RT chamber 31 and the PCR chamber 32 are formed in the same substrate 20 with a volume in a mini-scale, the analysis can be carried out using a very small amount of the sample and RT and PCR reagents. Thus, the time and cost needed to perform RT-PCR can be significantly reduced.

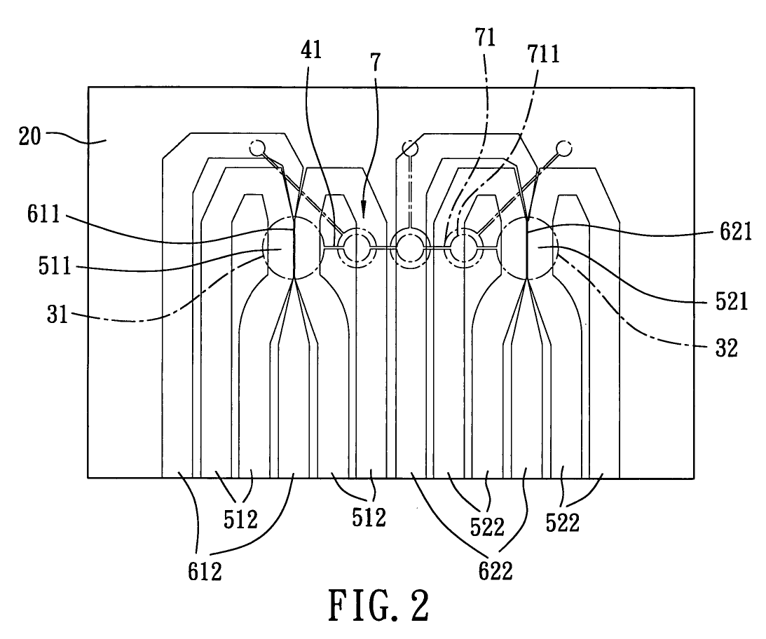

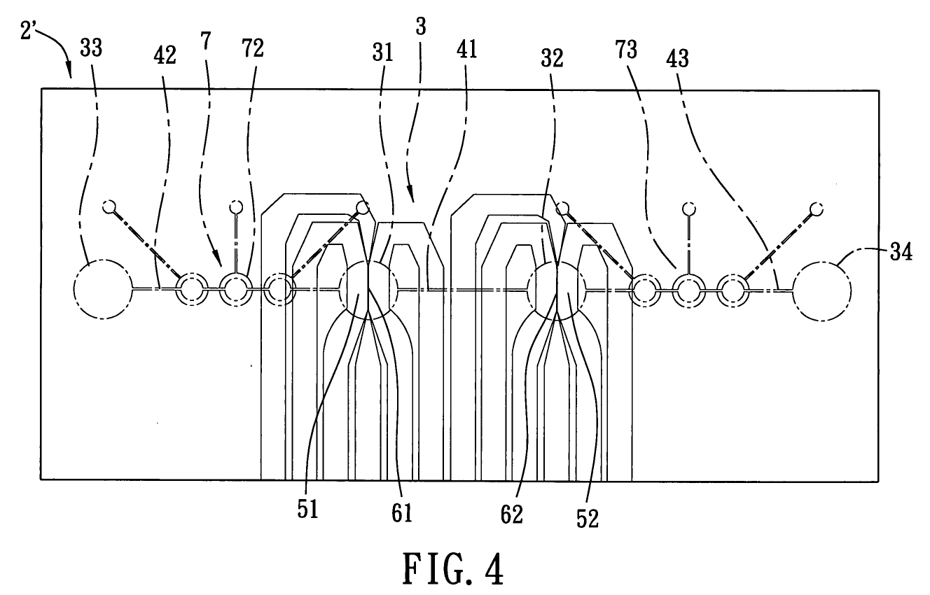

Referring to FIG. 4, there is shown a second preferred embodiment of the present invention which is substantially similar to the first preferred embodiment. However, the chip module 2′ in the second embodiment additionally includes an RT reagent storage 33, a PCR reagent storage 34, an RT reagent channel 42, and a PCR reagent channel 43. The RT reagent storage 33 is connected fluidly to the RT chamber 31 through the RT reagent channel 42. The PCR reagent storage 34 is connected fluidly to the PCR chamber 32 through the PCR reagent channel 43. No heating elements are provided to heat the RT and PCR reagent storages 33 and 34. In addition to the chamber channel pump 71 which controls the chamber channel 41 (shown in FIGS. 1 and 2), the pump unit 7 in this embodiment includes an RT reagent pump 72 and a PCR reagent pump 73 to control the RT reagent channel 42 and the PCR reagent channel 43, respectively. Each of the RT and PCR reagent pumps 72, 73 includes three valves 711 (see FIGS. 1 and 2).

In an example, a RNA virus-containing sample was injected into the RT chamber 31, the pump control unit 82 actuated the RT reagent pump 72 to open so that an RT reagent from the RT reagent storage 33 flowed into the RT chamber 31. The temperature control unit 81 controlled the first heating device 51, after the actuation of the RT reagent pump 72, to heat the RT chamber 31 so as to produce a complementary DNA. Thereafter, the pump control unit 82 actuated the chamber channel pump 71 in the chamber-to-chamber channel 41 to open so that the complementary DNA flowed into the PCR chamber 32. Subsequently, the chamber channel pump 71 was closed. At the same time, the PCR reagent pump 73 in the PCR reagent channel 43 was activated to permit a PCR reagent to flow into the PCR chamber 32, and the second heating device 52 was activated and controlled by the temperature control unit 81 to heat the PCR chamber 32. The operations of the first and second heating devices 51, 52 in this embodiment are similar to that described in the first embodiment.

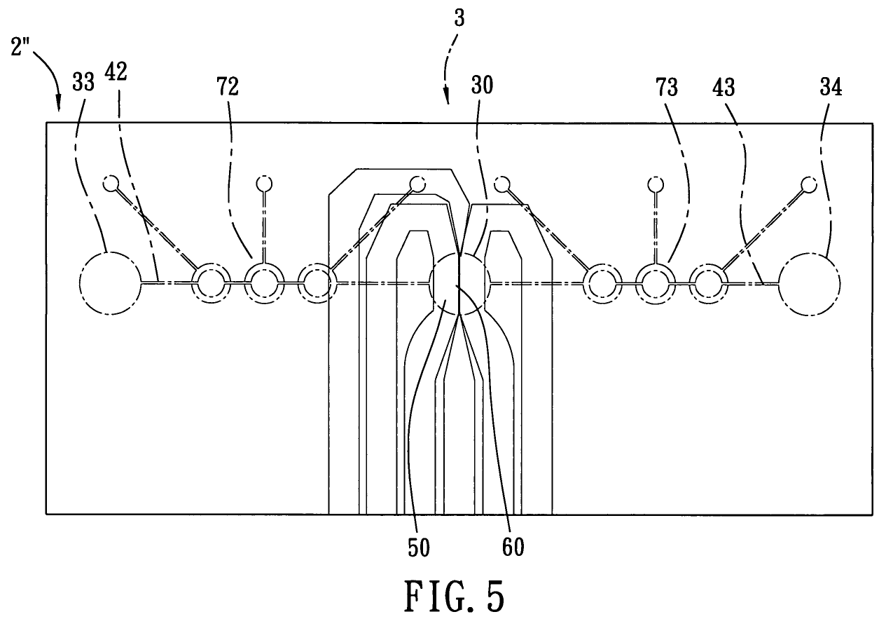

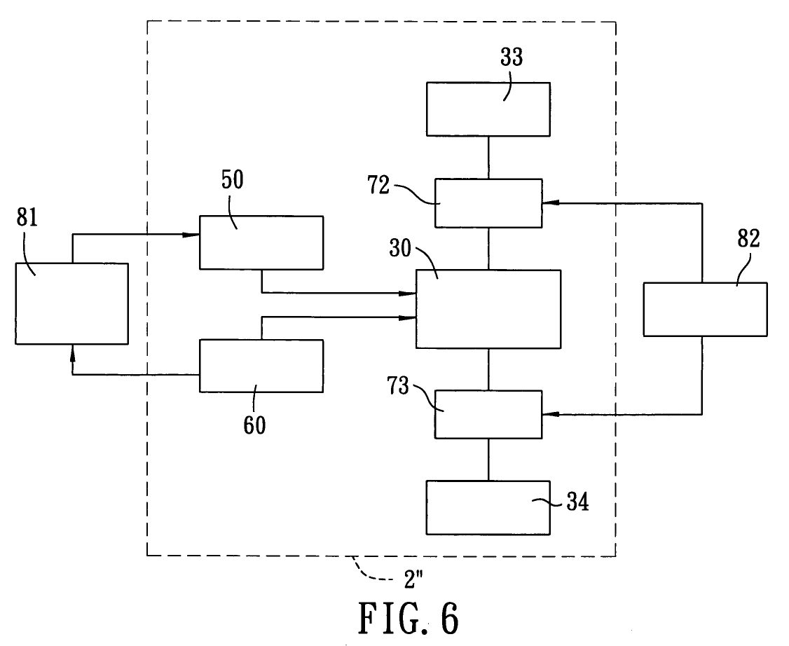

Referring to FIGS. 5 and 6, there is shown the third preferred embodiment of the present invention, which is substantially similar to the second embodiment of this invention, except for that the chip module 2″ includes a single reaction chamber 30 for conducting an RT-PCR reaction, a single heating device 50 for heating the reaction chamber 30, and a single temperature-sensing device 60. The reaction chamber 30 is connected fluidly to an RT reagent storage 33 through an RT reagent channel 42 and to a PCR reagent storage 34 through a PCR reagent storage 34. The RT reagent channel 42 is provided with the RT reagent pump 72, whereas the PCR reagent channel 43 is provided with the PCR reagent pump 73.

The temperature control unit 81 of the control module 8 controls the heating device 50 to perform the second heating operation after the first heating operation. The pump control unit 82 controls the RT channel pump 72 and the PCR channel pump 73. In particular, the pump control unit 82 actuates the RT channel pump 72 before the first heating operation so as to permit an RT reagent to flow from the RT reagent storage 33 to the reaction chamber 30 before the first heating operation. The pump control unit 82 further actuates the RT reagent pump 72 after the first heating operation so that a portion of a reaction product from the reaction chamber 30 flows to the RT reagent storage 33. After the RT reaction in the reaction chamber 30 through the first heating operation, the pump control unit 82 actuates the PCR reagent pump 73 to open so as to permit a PCR reagent to flow from the PCR reagent storage 34 to the reaction chamber 30.

In an example, an RNA virus-containing sample was injected into the reaction chamber 30, the pump control unit 82 actuated the RT reagent pump 72 to permit an RT reagent provided in the RT reagent storage 33 to flow into the reaction chamber 30. In cooperation with the temperature-sensing device 60, the temperature control unit 81 controlled the heating device 50 to perform a first heating operation so that the temperature of the reaction chamber 30 was raised to a level required to carry out the RT reaction. After a complementary DNA was formed in the reaction chamber 30, the pump control unit 82 actuated the RT reagent pump 72 to permit a predetermined amount of the complementary DNA to flow from the reaction chamber 30 to the RT reagent storage 33 so that a precise amount of the complementary DNA was left in the reaction chamber 30.

Thereafter, the pump control unit 82 actuated the PCR reagent pump 73 of the PCR reagent channel 43 to permit a PCR reagent to flow into the reaction chamber 30 from the PCR reagent storage 34. At this time, the temperature control unit 81 actuated the heating device 50 to perform the second heating operation for the PCR reaction. The first and second heating operations in this embodiment are the same as those described in the previous embodiments.

Referring once again to FIG. 1, the substrate 20 of the chip module 2 described hereinbefore includes an upper substrate part 22 and a lower substrate part 21. The substrate 20 may be manufactured through the following steps:

(A) Firstly, the lower substrate part 21 is manufactured by forming on a base layer a metal pattern which defines the first and second heating devices 51, 52 and the first and second temperature-sensing devices 61, 62 through microlithography and metal deposition techniques.

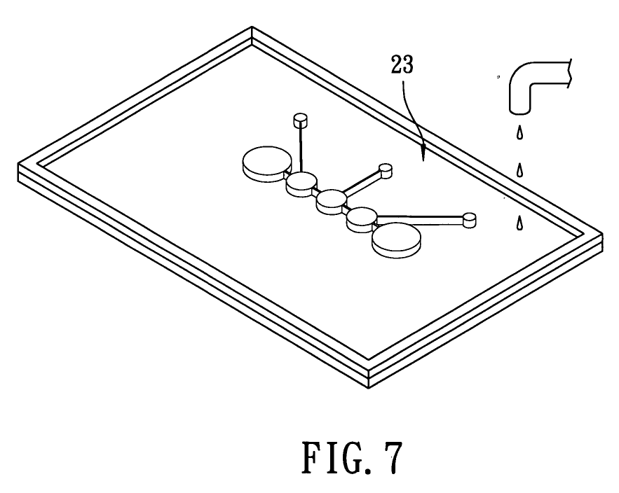

(B) Secondly, through microlithography and acid etching techniques, a female mold 23 (see FIG. 7) is fabricated by forming on another base layer a pattern that can impart the profiles of the chamber-to-chamber 4, the reaction chamber unit 3, and the pump unit 7. A polymeric molding material is then poured into the female mold 23 to form the upper substrate part 22.

(C) The upper substrate part 22 removed from the female mold 23 is stacked on and coupled with the lower substrate part 21 so that the RT and PCR chambers 31, 32 are disposed immediately above the first and second heating devices 51, 52, respectively.

As mentioned above, the heating unit 5 of the chip module 2, 2′ or 2″ is controlled by the control module 8 to heat the reaction chamber unit 3 to automatically perform the first and second heating operations for the reverse transcription-polymerase chain reaction. Therefore, by using the micro RT-PCR apparatus according to the present invention, an RT-PCR analysis may be carried out easily and quickly in a single chip module. In addition, the chip module 2, 2′ and 2″ can be mass-produced at a fast rate, thereby reducing production costs and time consumption.

While the present invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

We claim:

1. A micro reverse transcription-polymerase chain reaction (RT-PCR) apparatus, comprising:
a chip module including a reaction chamber unit, and a heating unit for heating said reaction chamber unit,
a control module including a temperature control unit for controlling said heating unit to perform a first heating operation and a second heating operation, said first heating operation providing said reaction chamber unit with a temperature required to carry out a reverse transcription (RT) reaction, said second heating operation providing said reaction chamber unit with a temperature required to perform a polymerase chain reaction (PCR).

2. The micro RT-PCR apparatus of claim 1, wherein said reaction chamber unit comprises an RT (reverse transcription) chamber, and a PCR (polymerase chain reaction) chamber in fluid communication with said RT chamber, said heating unit including a first heating device for heating said RT chamber during said first heating operation, and a second heating device for heating said PCR chamber during said second heating operation.

3. The micro RT-PCR apparatus of claim 2, wherein said chip module further comprises a temperature-sensing unit which includes a first temperature-sensing device disposed adjacent to said RT chamber and connected to said control module, and a second temperature-sensing device disposed adjacent to said PCR chamber and connected to said control module.

4. The micro RT-PCR apparatus of claim 3, wherein said chip module further comprises a chamber-to-chamber channel connected fluidly to said RT and PCR chambers, and a chamber channel pump associated with said chamber-to-chamber channel, said control module further including a pump control unit to actuate said chamber channel pump to open after said first heating operation.

5. The micro RT-PCR apparatus of claim 4, wherein said temperature control unit controls said second heating device to perform said second heating operation after said chamber channel pump opens.

6. The micro RT-PCR apparatus of claim 5, wherein said chip module further includes an RT reagent storage, a PCR reagent storage, an RT reagent channel interconnecting said RT reagent storage and said RT chamber, and a PCR reagent channel interconnecting said PCR chamber and said PCR reagent storage.

7. The micro RT-PCR apparatus of claim 6, wherein said chip module further includes an RT reagent pump and a PCR reagent pump associated with said RT and PCR reagent channels, respectively.

8. The micro RT-PCR apparatus of claim 1, wherein said reaction chamber unit includes a single reaction chamber, said heating unit including a single heating device to heat said reaction chamber.

9. The micro RT-PCR apparatus of claim 8, wherein said chip module further includes an RT reagent storage, a PCR reagent storage, an RT reagent channel interconnecting said RT reagent storage and said reaction chamber, and a PCR reagent channel interconnecting said PCR reagent storage and said reaction chamber.

10. The micro RT-PCR apparatus of claim 9, wherein said chip module further includes an RT reagent pump and a PCR reagent pump which are associated with said RT and PCR reagent channels, respectively.

11. The micro RT-PCR apparatus of claim 10, wherein said control module further includes a pump control unit to actuate said RT reagent pump and said PCR reagent pump.

12. The micro RT-PCR apparatus of claim 11, wherein said pump control unit actuates said PCR reagent pump to open after said RT reagent pump is actuated by said pump control unit.

13. An RT-PCR method comprising:
using a chip module which has a reaction chamber unit and a heating unit for heating said reaction chamber unit;
carrying out an RT reaction and a PCR reaction in said reaction chamber unit; and
controlling said heating unit to perform a first heating operation and a second heating operation, said first heating operation providing said reaction chamber unit with a temperature required to carry out the RT reaction, said second heating operation providing said reaction chamber unit with a temperature required to perform the PCR reaction.

14. The RT-PCR method according to claim 13, further comprising:
providing an RT chamber and a PCR chamber that constitute said reaction chamber unit;
carrying out the RT reaction in said RT chamber through said first heating operation;
permitting a reaction product to flow from said RT chamber to said PCR chamber after said first heating operation; and carrying out the PCR reaction in said PCR chamber through said second heating operation.

15. The RT-PCR method according to claim 14, further comprising:
providing said chip module with an RT reagent storage and a PCR reagent storage which are connected fluidly to said RT chamber and said PCR chamber, respectively; permitting an RT reagent to flow from said RT reagent storage to said RT chamber for the RT reaction; and permitting a PCR reagent to flow from said PCR reagent storage to said PCR chamber for the PCR reaction.

16. The RT-PCR method according to claim 13, wherein said reaction chamber unit includes a single reaction chamber, the RT and PCR reactions being carried out in said reaction chamber through said first and second heating operations.

17. The RT-PCR method according to claim 16, further comprising:
providing said chip module with an RT reagent storage and a PCR reagent storage which are connected fluidly to said reaction chamber;
permitting an RT reagent to flow from said RT reagent storage to said reaction chamber to carry out the RT reaction; and
permitting a portion of a reaction product to flow from said reaction chamber to said RT reagent storage after the RT reaction and maintaining a remaining portion of said reaction product in said reaction chamber;
permitting a PCR reagent to flow from said PCR reagent storage to said reaction chamber so that said PCR reagent and said remaining portion undergo the PCR reaction in said reaction chamber.
